(19) United States
(12) Patent Application Publication
(10) Pub. No.: US 2024/0302337 A1
(43) Pub. Date: Sep. 12, 2024

(54) ACCURATE SPECTRAL LIBRARY SEARCH

(71) Applicant: CERNO BIOSCIENCE LLC, Las Vegas, NV (US)

(72) Inventors: Yondong Wang, Las Vegas, NV (US); Stacey Siminoff, Portsmouth, NH (US); Don Kuehl, Windham, NH (US)

(73) Assignee: CERNO BIOSCIENCE LLC, Las Vegas, NV (US)

(21) Appl. No.: 18/647,333

(22) Filed: Apr. 26, 2024

Related U.S. Application Data
(63) Continuation of application No. PCT/US2022/048228, filed on Oct. 28, 2022.
(60) Provisional application No. 63/273,676, filed on Oct. 29, 2021.

(51) Int. Cl. G01N 30/86

(52) U.S. Cl. CPC G01N 30/8651; G01N 30/8637; G01N 30/8665

(57) ABSTRACT

A method, mass spectrometer and computer readable medium for acquiring mass spectral data; comprising acquiring mass spectral data for a sample in a profile mode; computing spectral loadings from the acquired mass spectral data; operating with the spectral loadings and a mass spectral library spectrum in profile mode to calculate a search score for a library compound; and reporting search scores indicating the likelihood of library compounds being present in the sample. A regression analysis is performed to estimate the relative concentrations of possible compounds. The method can be implemented in the form of a server located amongst networks, such as the worldwide web, computers, various devices, and MS instruments. Users of the method, while waiting for results, are exposed to advertising. The advertising is selected to be relevant to the compounds being analyzed. Users of the method are provided with a subscription to updates in the library.

The present application is a continuation of International Patent Application PCT/US2022/048228, of the same title and filed on Oct. 28, 2022, which claims priority to U.S. Provisional Patent Application No. 63/273,676, also of the same title and filed on Oct. 29, 2021, the entirety of each which are incorporated herein by reference, in their entireties, for all purposes.

### CROSS REFERENCE TO RELATED PATENT APPLICATIONS/PATENTS

U.S. Pat. Nos. 6,983,213, 7,493,225 and 7,577,538; International Patent Application PCT/US2004/013096, filed on Apr. 28, 2004; U.S. Pat. No. 7,348,553; International Patent Application PCT/US2005/039186, filed on Oct. 28, 2005; U.S. Pat. No. 8,010,306, International Patent Application PCT/US2006/013723, filed on Apr. 11, 2006; U.S. Pat. No. 7,781,729, International Patent Application PCT/US2007/069832, filed on May 28, 2007; U.S. provisional patent application Ser. No. 60/941,656, filed on Jun. 2, 2007, and as International Patent Application PCT/US2008/065568 published as WO 2008/151153; and U.S. provisional patent application Ser. No. 62/830,832, filed on Apr. 8, 2019 and as U.S. patent application Ser. No. 16/843,505 published as US 2020-0232956 A1.

The entire teachings of these patent documents are hereby incorporated herein by reference, in their entireties, for all purposes.

### FIELD OF THE INVENTION

The present invention generally relates to the field of Mass Spectrometry (MS) and, more particularly, to methods for acquiring, processing, and analyzing MS data. The same approach is also applicable to other spectroscopic or spectrometric technologies such as infrared (IR), ultraviolet, visible, fluorescence, and Raman, especially when used in combination with a separation technique such as chromatography.

### BACKGROUND OF THE INVENTION

Mass Spectrometry (MS) is 100-year-old technology that relies on the ionization of molecules, the dispersion of the ions by their masses, and the proper detection of the ions on the appropriate detectors. There are many ways to achieve each of these three key MS processes which give rise to different types of MS instrumentations having distinct characteristics.

Many ionization techniques are available to ionize molecules entering MS system so that they can be properly charged before mass dispersion. These ionization schemes include Electrospray Ionization (ESI), Electron Impact Ionization (EI) through the impact of high-energy electrons, Chemical Ionization (CI) through the use of reactive compounds, and Matrix-Assisted Laser Desorption and Ionization (MALDI).

Once the molecules have been charged through ionization, each ion will have a corresponding mass-to-charge (m/z) ratio, which will become the basis for mass dispersion. Based on the physical principles used, there are many different ways to achieve mass dispersion and subsequent ion detection, resulting in mass spectral data similar in nature but different in details. A few of the commonly seen configurations include: magnetic/electric sector; quadrupoles; Time-Of-Flight (TOF); and Fourier Transform Ion-Cyclotron Resonance (FT ICR).

The sector MS configuration is the most straight-forward mass dispersion technique where ions with different m/z ratios separate in an electric/magnetic field and exit this field at spatially separated locations where they will be detected with either a fixed array of detector elements or a movable set of small detectors that can be adjusted to detect different ions depending on the application. This is a simultaneous configuration where all ions from the sample are separated simultaneously in space rather than sequentially in time.

The quadrupoles configuration is perhaps the most common MS configuration where ions of different m/z values are filtered out of a set of (usually 4) parallel rods through the manipulation of RF/DC ratios applied to these rod pairs. Only ions of a certain m/z value will survive the trip through these rods at a given RF/DC ratio, resulting in the sequential separation and detection of ions. Due to its sequential nature, only one detector element is required for detection. Another configuration that uses ion traps can be conceptually considered a special example of a quadrupole MS.

The Time-Of-Flight (TOF) configuration is another sequential dispersion and detection scheme that lets ions enter through a high vacuum flight tube before detection. Ions of different m/z values arrive at different times at the detector and the arrival time can be related to the m/z values through the use of known calibration standard(s). In Fourier Transform Ion-Cyclotron Resonance (FT ICR), all ions can be introduced to an ion cyclotron where ions of different m/z ratios would be trapped and resonate at different frequencies. These ions can be pulsed out through the application of a Radio Frequency (RF) signal and the ion intensities measured as a function of time on a detector. Upon Fourier transformation of the time domain data measured, one gets back the frequency domain data where the frequency can be related back to m/z through the use of known calibration standard(s). Orbitrap MS systems can be conceptually considered as a special case of FT MS.

As discussed in the cross-referenced U.S. Pat. No. 6,983,213, a mass spectral data trace is typically subjected to peak analysis where peaks (ions) are identified. This peak detection routine is a highly empirical and compounded process where peak shoulders, noise in data trace, baselines due to chemical backgrounds or contamination, isotope peak interferences, etc., are considered. For the peaks identified, a process called centroiding is typically applied to report only two data values, m/z location and estimated peak area (or peak height), wherever an MS peak is detected. While highly efficient in terms of data storage, this is a process plagued by many adjustable parameters that can make an isotope seem to appear or disappear with no objective measures of the centroiding quality, due to the many interfering factors mentioned above and the intrinsic difficulties in determining peak areas in the presence of other peaks and/or baselines. Unfortunately for many MS systems, especially quadrupole MS systems, this MS peak detection and centroiding are conventionally set up by default, as part of the MS method, to occur during data acquisition, at the firmware level. This leads to irreparable damages to the MS data integrity, even for pure component mass spectral data in the absence of any spectral interferences from other co-existing compounds or analytes. As pointed out in U.S. Pat. No. 6,983,213, these damages or disadvantages include:
a. Lack of mass accuracy on the most commonly used unit mass resolution MS systems. The centroiding process forces the reported mass value into integer m/z with ±1 Da or other m/z values with at least ±0.1 Da mass error, whereas the properly calibrated raw profile mode MS data (without centroiding) using the method disclosed in U.S. Pat. No. 6,983,213 can be accurate to +0.005 Da, a factor of approximately 100 improvement.
b. Large peak integration error. Centroiding without full mass spectral calibration including MS peak shape calibration suffers from uncertainty in mass spectral peak shape, its variability, the isotope peaks, the baseline and other background signals, with random noise, leading to both systematic and random errors for either strong or weak mass spectral peaks.
c. Large isotope abundance error. Separating the contributions from various closely located isotopes (e.g., A and A+1) on conventional MS systems with unit mass resolution either ignores the contributions from neighboring isotope peaks or over-estimates them, resulting in errors for dominating isotope peaks and large biases for weak isotope peaks or even complete elimination of the weaker isotopes.
d. Loss of Linear Additivity. For overlapping mixture peaks, the centroiding would have to force a peak into the nearest integer position, creating a quantized mass location error depending on the relative amount of the overlapped components. As the relative amounts vary across a chromatographic peak, the centroided peak area may be associated with different integer masses, destroying the linearly additive nature of the MS signal existing in the profile mode data.
e. Nonlinear operation. The centroiding typically uses a multi-stage disjointed process with many empirically adjustable parameters during each stage. Systematic errors (biases) are generated at each stage and propagated down to the later stages in an uncontrolled, unpredictable, and nonlinear manner, making it impossible for the algorithms to report meaningful statistics as measures of data processing quality and reliability.
f. Dominating systematic errors. In most of MS applications, ranging from industrial process control and environmental monitoring to protein identification or biomarker discovery, instrument sensitivity or detection limit has always been a focus and great efforts have been made in many instrument systems to minimize measurement error or noise contribution in the signal. Unfortunately, the typical centroiding process currently in use create a source of systematic error even larger than the random noise in the raw data, thus becoming the limiting factor in instrument sensitivity.
g. Mathematical and statistical inconsistency. The many empirical approaches currently used in centroiding make the whole processing inconsistent either mathematically or statistically. The peak processing results can change dramatically on slightly different data without any random noise or on the same synthetic data with slightly different noise. In order words, the results of the peak centroiding are not robust and can be unstable depending on a particular experiment or data acquisition.
h. Instrument-to-instrument or tune-to-tune variability. It has usually been difficult to directly compare raw mass spectral data from different MS instruments due to variations in the mechanical, electromagnetic, or environmental tolerances. The typical centroiding applied to the actual raw profile mode MS data, not only adds to the difficulty of quantitatively comparing results from different MS instruments due to the quantized nature of the centroiding process and centroid data, but also makes it difficult, if not impossible, to track down the source or possible cause of the variability once the MS data have been reduced to centroid data.

For a well separated analyte with pure mass spectrum and without any spectral interferences, MS centroiding is quite problematic, for the above listed reasons. For unresolved or otherwise co-eluting analytes or compounds in complex samples (e.g., petroleum products or essential oils) even after extensive chromatographic separation (e.g., 1-hour GC separation of essential oils or elaborate 1-2 hour(s) LC separation of biological samples with post translational modification such as deamidation), the above centroid processing problem would only be further aggravated due to the mutual mass spectral interferences present and the quantized nature of the MS centroids, which makes mass spectral data no longer linearly additive. This necessarily makes the MS centroid spectrum of a mixture different from the sum of MS centroids obtained from each individual pure spectrum, thus making the nonlinear and systematic centroiding error worse and even intractable. For this reason, the conventional co-elution deconvolution approach in common use, called AMDIS (Automated Mass Spectral Deconvolution & Identification System) as reported in “Optimization and Testing of Mass Spectral Library Search Algorithms for Compound Identification” Stein, S. E.; Scott, D. R. J. Amer. Soc. Mass Spectrom. 1994, 5, 859-866, which typically operates with MS centroid data, often fails to determine the correct number of co-elution compounds, derive the correct separation time profiles (called chromatograms in the case of chromatographic separation) of individual compounds or analytes, or compute the correct pure component/analyte mass spectra for reliable library (e.g., NIST EI MS library) search and compound identification.

For complex samples without any time-based (e.g., chromatographic) separation due to the need for speedy analysis or detection, using, as an example, novel ionization techniques such as DART (Direct Analysis in Real Time), reported in R. B. Cody; J. A. Laramée; H. D. Durst (2005) “Versatile New Ion Source for the Analysis of Materials in Open Air under Ambient Conditions”. Anal. Chem. 77 (8): 2297-2302, the mass spectrum may become so complex that there may not be visually separable mass spectral peaks for either detection or centroiding, possibly leading to the outright total failure of conventional mass spectral data acquisition, processing, and analysis.

Further compounding all the problems associated with mass spectral centroiding during a test sample analysis, nearly all established mass spectral libraries (e.g., NIST or Wiley libraries) have been created in the centroid mode, leading to another sources of errors, uncertainties, and undesirable nonlinear behaviors during the spectral library search process for either compound identification or quantitative analysis. Due to the sheer number (more than 100,000's) of pure compounds involved and many decades of detailed work, careful experimentation, and measurements in creating, maintaining, confirming and updating these libraries, it is very difficult or impractical to recreate these existing libraries in accurate profile mode.

Accordingly, it would be desirable and highly advantageous to have methods to avoid MS peak detection and centroiding altogether to overcome the above-described deficiencies and disadvantages of the prior art, for both real sample analysis and, most significantly, for creating accurate profile mode mass spectral libraries, to initially enhance and eventually replace the centroid mode mass spectral libraries currently in wide use.

Additionally, while more information is preserved in the profile mode data, the library search in the profile mode presents a unique set of challenges due to the 10-15 times the extra data points involved in each spectrum. There are over 330,000 spectra in the current version of the NIST spectral library. Even for the centroid mode spectral library search, various schemes such as pre-filtering have to be used in order to make the search on a regular computer fast enough to be practical. Such schemes come with some well researched risks, especially in the presence of spectral interferences or in the event of co-eluting compounds, where a correct compound may be assigned a much compromised search score and therefore not appear among the limited number of top hits to be even considered as a possible candidate.

### SUMMARY OF THE INVENTION

The present application is directed to the following improvements:
1. While integer centroid searches can easily match peaks by computing a library dot product, accurate mass centroid data search using HiRes TOF or Obitrap MS is difficult and error-prone because there must be a mass tolerance window specified. Profile searches eliminate the need to judge whether centroids match by operating on all spectrum points. Moreover, profile search using dot products lends itself to modern computer parallelism including SIMD (Single Instruction Multiple Data) instructions, GPUs, and multicore CPUs (https://en.wikipedia.org/wiki/Streaming_SIMD_Extensions). This allows it to be as fast as, or faster than, even integer search algorithms which must include difficult-to-optimize branch instructions in the inner computation loop. This profile search approach maintains accurate mass information automatically without any user-specified mass tolerance window.
2. A computationally effective approach to perform profile mode mass spectral library searches, by taking advantage of the highly parallel features of modern computers and the characteristics of mass spectral data and devising efficient numerical methods.
3. A new general-purpose approach to mixture analysis and identifying compounds that co-elute with each other, by taking advantage of the accuracy intrinsic in the profile mode data and particularly profile data after accurate mass and spectral accuracy calibration.
4. An approach to leverage current commercial libraries to create a profile mode library to jump start the Accurate Mass Profile Search (AMPS) library and gradually improve and refine it through the reliable and accurate identification of many compounds contained in a complex test sample during routine analysis, towards the eventual goal of complete mass spectral libraries containing accurate profile mode spectral data.
5. A server- or cloud-based implementation to both speed up individual searches and scale up the library refinement efforts.

Each of these aspects will be described below along with experimental results to demonstrate their utilities.

### BRIEF DESCRIPTION OF THE DRAWINGS

FIG. 1 is a block diagram of a mass spectrometer system that can utilize the methods disclosed herein.

FIG. 2A and FIG. 2B are two graphs of the mass spectra obtained from the same compound on three different GC/MS instruments, where the top graph shows the raw mass spectra as measured and the bottom graph shows the same after accurate mass and spectral accuracy calibration.

FIG. 3 shows a segment of the Total Ion Chromatogram (TIC) obtained from a GC/MS analysis of a sample containing Volatile Organic Compounds (VOCs).

FIG. 4A and FIG. 4B show a plot of sorted search scores using the approach disclosed herein, where the bottom is a zoomed-in version of the top graph showing the top 20 hits.

FIG. 5 includes a flow chart of one embodiment disclosed herein.

A component or a feature that is common to more than one drawing is indicated with the same reference number in each of the drawings.

### DETAILED DESCRIPTION OF THE PREFERRED EMBODIMENTS

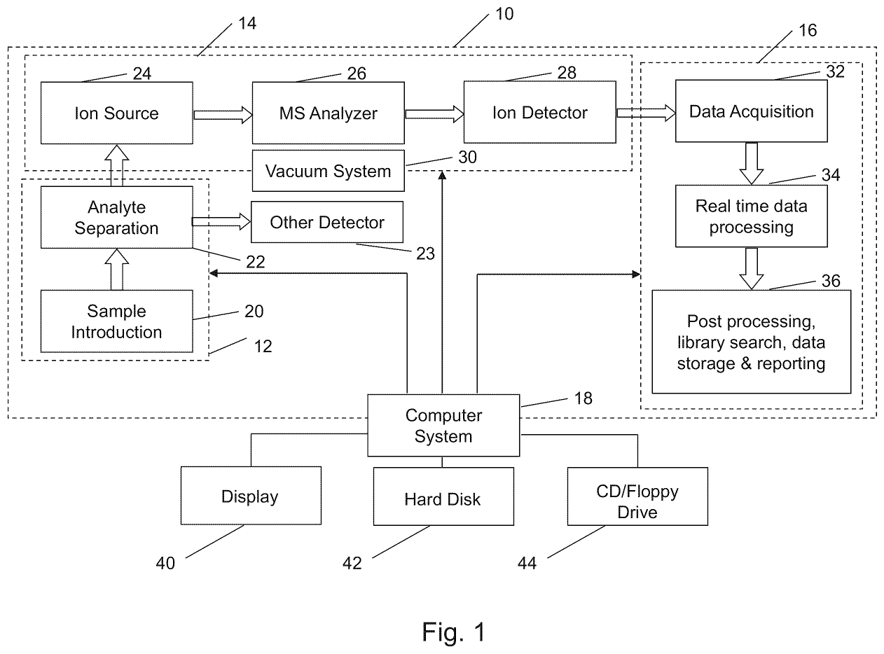

Referring to FIG. 1, there is shown a block diagram of an analysis system 10, that may be used to analyze proteins or other molecules, as noted above, incorporating features of the apparatus and methods disclosed herein. Although the present invention will be described with reference to the single embodiment shown in the drawings, it should be understood that the present invention can be embodied in many alternate forms of embodiments. In addition, any suitable types of components could be used.

Analysis system 10 has a sample preparation portion 12, other detector portion 23, a mass spectrometer portion 14, a data analysis system 16, and a computer system 18. The sample preparation portion 12 may include a sample introduction unit 20, of the type that introduces a sample containing proteins, peptides, or small molecule drugs of interest to system 10, such as LCQ Deca XP Max, manufactured by Thermo Fisher Scientific Corporation of Waltham, MA, USA. The sample preparation portion 12 may also include an analyte separation unit 22, which is used to perform a preliminary separation of analytes, such as the proteins to be analyzed by system 10. Analyte separation unit 22 may be any one of a chromatography column, an electrophoresis separation unit, such as a gel-based separation unit manufactured by Bio-Rad Laboratories, Inc. of Hercules, CA, or other separation apparatus such as ion mobility or pyrolysis etc. as is well known in the art. In electrophoresis, a voltage is applied to the unit to cause the proteins to be separated as a function of one or more variables, such as migration speed through a capillary tube, isoelectric focusing point (Hannesh, S. M., Electrophoresis 21, 1202-1209 (2000), or by mass (one dimensional separation)) or by more than one of these variables such as by isoelectric focusing and by mass. An example of the latter is known as two-dimensional electrophoresis.

The mass spectrometer portion 14 may be a conventional mass spectrometer and may be any one available, but is preferably one of TOF, quadrupole MS, ion trap MS, qTOF, TOF/TOF, or FTMS. If it has an electrospray ionization (ESI) ion source, such ion source may also provide for sample input to the mass spectrometer portion 14. In general, mass spectrometer portion 14 may include an ion source 24, a mass analyzer 26 for separating ions generated by ion source 24 by mass to charge ratio, an ion detector portion 28 for detecting the ions from mass analyzer 26, and a vacuum system 30 for maintaining a sufficient vacuum for mass spectrometer portion 14 to operate most effectively. If mass spectrometer portion 14 is an ion mobility spectrometer, generally no vacuum system is needed and the data generated are typically called a plasmagram instead of a mass spectrum.

In parallel to the mass spectrometer portion 14, there may be an other detector portion 23, where a portion of the flow is diverted for nearly parallel detection of the sample in a split flow arrangement. This other detector portion 23 may be a single channel UV detector, a multi-channel UV spectrometer, or Reflective Index (RI) detector, light scattering detector, radioactivity monitor (RAM) etc. RAM is most widely used in drug metabolism research for 14C-labeled experiments where the various metabolites can be traced in near real time and correlated to the mass spectral scans.

The data analysis system 16 includes a data acquisition portion 32, which may include one or a series of analog to digital converters (not shown) for converting signals from ion detector portion 28 into digital data. This digital data is provided to a real time data processing portion 34, which processes the digital data through operations such as summing and/or averaging. A post processing portion 36 may be used to do additional processing of the data from real time data processing portion 34, including library searches, data storage and data reporting.

Computer system 18 provides control of sample preparation portion 12, mass spectrometer portion 14, other detector portion 23, and data analysis system 16, in the manner described below. Computer system 18 may have a conventional computer monitor or display 40 to allow for the entry of data on appropriate screen displays (using, for example, a keyboard, not shown), and for the display of the results of the analyses performed. Computer system 18 may be based on any appropriate personal computer, operating for example with a Windows® or UNIX® operating system, or any other appropriate operating system. Computer system 18 will typically have a hard drive 42 or other type of data storage medium, on which the operating system and the program for performing the data analysis described below, is stored. A removable data storage device 44 for accepting a CD, floppy disk, memory stick or other data storage medium is used to load the program in accordance with the invention on to computer system 18. The program for controlling sample preparation portion 12 and mass spectrometer portion 14 will typically be downloaded as firmware for these portions of system 10. Data analysis system 16 may be a program written to implement the processing steps discussed below, in any of several programming languages such as C++, JAVA or Visual Basic.

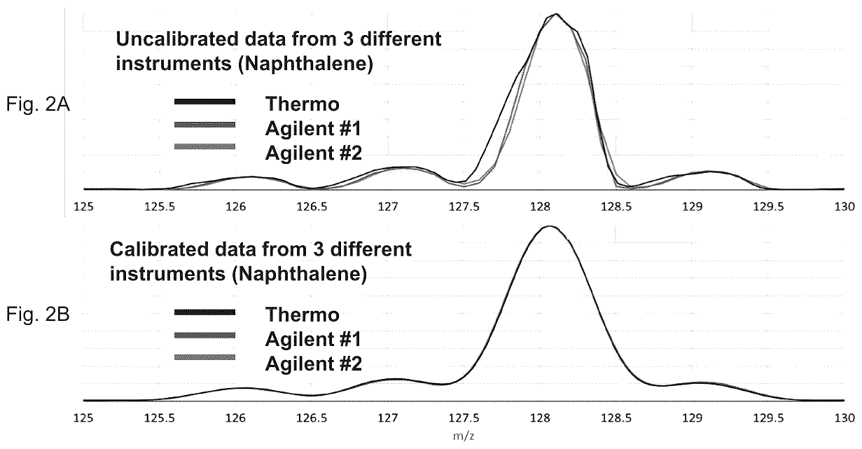

In the preferred embodiment of this invention, a sample is acquired through the chromatography/mass spectrometry system described in FIG. 1 with mass spectral profile mode raw data continuously acquired throughout the run, resulting in a data run with typical raw profile mode mass spectra such as the ones shown in FIG. 2A, which can be calibrated for both mass accuracy and spectral accuracy, resulting in the highly accurate and consistent calibrated spectra shown in FIG. 2B. This accurate and comprehensive calibration is performed before subsequent processing and analysis, using the approach described in the U.S. Pat. No. 6,983,213. Such a calibration not only calibrates for mass accuracy and spectral accuracy, but also achieves a significant degree of noise filtering, which is quite important or even critical for compound identification at low concentration levels approaching the detection limit.

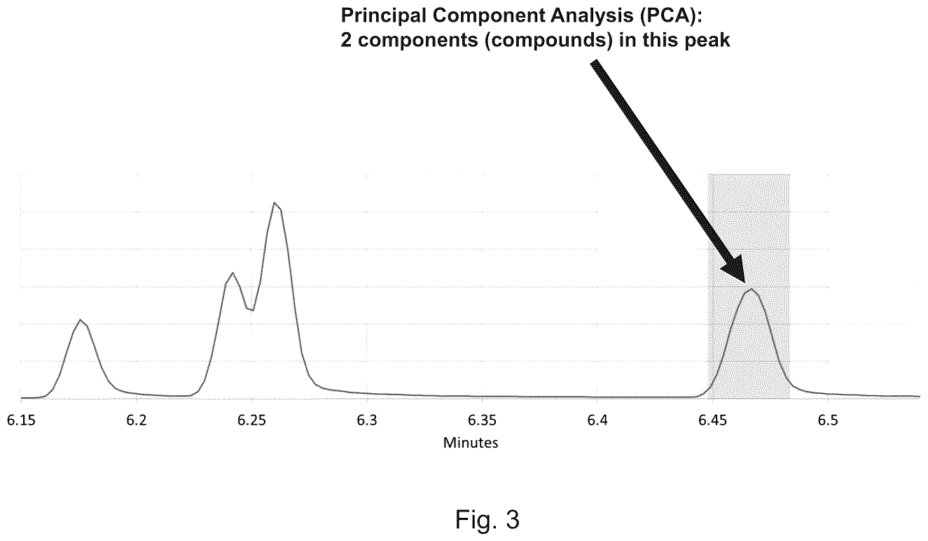

The detailed steps involved in the subsequent processing and analysis would now be described:
a. Detection of all the chromatographic peaks from the total ion chromatogram (TIC) shown in in FIG. 3. This can be best accomplished with known pure chromatographic peak shape functions across the whole separation time range, which can be measured under the same chromatographic separation conditions using a set of known standards such as alkane with different carbon numbers to cover the required retention time range. One may also perform a chromatographic peak shape calibration to convert the actual peak shape into target peak shape, much like how mass spectral peak shape calibration is performed in U.S. Pat. No. 6,983,213 and further disclosed in the U.S. patent application Ser. No. 11/402,238, filed on Apr. 10, 2006. Once the chromatographic peak shape is well defined through either actual measurement or calibration, the peak detection and analysis method from U.S. Pat. No. 6,983,213 can be utilized to detect all chromatographic peaks in a chromatogram such as the shaded region shown in FIG. 3.
b. Some of the detected peaks are pure and therefore ready for library search (identification) or quantitative analysis but some of which are not pure and would not be directly suitable for either. It is critical to identify these chromatographic peaks to assess their purity. In order to achieve purity detection as well as the reliable mixture search to follow, it is imperative to have a reliable approach for the determination of independent analytes contained in a chromatographic peak or separation time window. This is accomplished by performing multivariate statistical analysis on the acquired profile mode mass spectral scan data corresponding to the separation time window. The multivariate statistical analysis can be accomplished using a variety of well established algorithms known in the art, such as Principal Component Analysis (PCA) or Partial Least Squares, based on either Singular Value Decomposition or NIPALS algorithm (S. Wold, P. Geladi, K. Esbensen, J. Öhman, J. Chemometrics, 1987, 1(1), 41).
c. Once the correct number of components are determined (two components shown for the shaded peak in FIG. 3), the next step is to perform compound identification using the AMPS. As mentioned earlier, profile mode mass spectrum after calibration for mass accuracy and spectral accuracy is much preferred here due to both signal to noise enhancement and better accuracy in subsequent processing. Take full profile mode MS spectral data across any GC peak (m time points and n m/z values) and perform PCA analysis

D(m×n)=U(m×p)S(p×p)V′(p×n)

where p is the number of principal components found, U are scores and V are the loadings. A projection matrix can be constructed as:

$$P ( nxn ) = V ( nxp ) N ’ ( pxn ) \qquad ( Eq 1 )$$

Pick any library spectrum I from the huge library and project it onto the p-component subspace to obtain a projected version of the library spectrum

$$1 \_ ( nxl ) = P ( nxn ) 1 ( nxl ) \qquad ( Eq 2 )$$

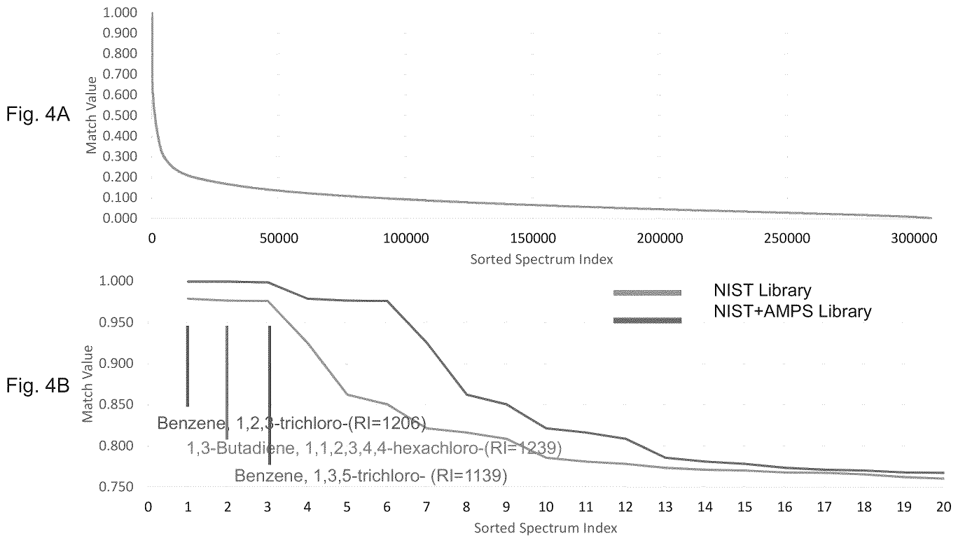

While conceptually feasible, the above Eq 1 and 2 can be computationally expensive, since it involves a huge projection matrix of n×n where n could reach 10,000 m/z values to be applied to over 300,000 library spectra. A computationally much more efficient alternative is to write out the projection as I_(n×1)=P (n×n) I (n×1)=V(n×p) V′(p×n) I (n×1)=V(n×p) [V′(p×n) \(n×1)] where [V′(p×n) \ (n×1)] is the dot product search of each of p loadings with each of the 300,000 spectra in the library, resulting in p dot products for each library spectrum I. These p dot products are then efficiently used as combination coefficients to linearly combine with the p loadings in V(n×p) to produce a projected version of the library spectrum I_. The computation cost in this case is linear with the number of components p, i.e., p times the typical dot product search.
d. If a library spectrum is indeed contained in the p-component subspace (i.e., the corresponding compound is part of the mixture in question), its projection onto the subspace would leave it unchanged, subject only to random noise or any modeling error, i.e., its length before and after projection would be the same. Otherwise, the projected version could only have a shorter length. The ratio of the length after and before the projection would be the search or match score indicating whether the compound corresponding to the particular library spectrum is present in this chromatographic peak. Such a search score can be obtained for all spectra in the library and all scores can be sorted from high to low, as plotted in FIG. 4.
e. Perform a Multiple Linear Regression (MLR) analysis between the acquired profile mode mass spectrum and those with high search scores from the above described library search to obtain relative concentrations of possible compounds contained in the chromatographic peak and report relevant statistics such as t-values to indicate the significance of estimated relative concentrations which may point to the presence or absence of possible compounds.
f. Report the compounds identified and their relative concentrations, where the relative concentrations can be further converted into absolute quantitative results using known concentration standards or standard series, or semi-quantitative results by ratioing against other internal or external reference standards or ions.
g. Compounds positively identified with high confidence both in terms of signal to noise (concentration) and purity would at least rival those isolated and produced in pure form from a lab or purchased from a commercial supplier such as Sigma-Aldrich. Their (preferably calibrated, accurate mass) profile mode mass spectra qualify to be entered into a library as library spectra for future search and unknown or test sample analysis, by either replacing or augmenting an existing profile mode library converted from centroid library through convolution with a given peak shape function. Pure compounds that could not be confidently identified from the library but with high signal-to-noise spectra may be true unknowns that have not been measured before and may be entered in a library as such, pending further information to be added, such as structure, elemental composition, CAS, etc. There are multiple advantages associated with this new approach:
   i. This is a highly efficient way to generate a profile mode mass spectral library since a complex test sample may contain as many as 100-200 compounds at enough concentrations which are separated automatically during either the GC separation or the co-elution deconvolution through the post-acquisition GC/MS analysis described above. Thus, at least 100 qualified library spectra corresponding to 100 individual compounds including the hard-to-obtain-or-separate isomers could be measured in a single GC/MS experiment, as opposed to 100 separate GC/MS runs with commercially purchased pure standards, saving not only a tremendous amount of time but also huge associated expenses, while avoiding nearly all human errors during the long painstaking experimentation that otherwise would be needed.
   ii. The isolated pure standards purchased may not be stable by themselves and would require certain stable solutions for them to be stored in, requiring extra storage space and sample preparation before each GC/MS analysis.
   iii. A different GC/MS analysis method may have to be developed individually and specifically for some standards, further adding to the challenges and workloads.
   iv. Some standards may not be available in pure form at all or may not be stable enough to be measured alone.
   v. Instead of human inspection and curation of library spectra, this new approach automatically checks the quality of the measurement by treating a compound as an unknown compound to be identified, saving time and efforts while avoiding human errors.
   vi. Re-measuring and doubling-checking previously measured library spectra would require the long-term storage of many thousands of compounds, each of which would have its own lifetime, presenting huge informatics and logistics challenges. This new approach of generating qualified library spectra through actual complex sample analysis allows for the same compound to be detected and measured time and again in a sample containing the compound, providing an opportunity to compare with previously measured library spectra via one or more of the available library match score, mass accuracy, spectral accuracy, retention index match, and possible fragment analysis, thus allowing for the library to dynamically improve upon itself over time by always keeping the best library spectra in the library.
   vii. In the case where the new profile mode mass spectra data are added to an existing mass spectral library converted from a centroid library, the library search immediately benefits from these compounds with profile mode mass spectral data when one of these compounds are found to have both centroid-converted profile mode data and the new more accurate profile mode mass spectral data. Such a living and ever improving library adds extra value from the very beginning and continues improving upon itself, while continuing with actual real world test sample analysis. It is expected that eventually all centroid-converted mass spectral data would be replaced with the more accurate (preferably accurate mass and spectral accuracy calibrated) profile mode data. One could imagine a commercial business where each year, quarter, or month, a newer and progressively more accurate library could be released to end users for a fee.
   viii. By operating in tandem with existing centroid-converted profile mode libraries, one has the benefit of being able to take advantage of all other existing information related to a compound, including trade names, synonyms, structures, retention index, CAS number, which have already been carefully curated and checked by generations of scientists and technicians.
   ix. It is feasible to implement this approach in the form of a Web or cloud server where any end user from around the globe could submit a prescribed measurement run data, preferably with both retention time standard (e.g., n-alkane) and MS calibration standard (e.g., PFTBA) included, for actual real time analysis of real test samples. The more accurate profile mode mass spectra for compounds identified with high confidence can be stored in the library for future use, to replace the corresponding centroid-converted library spectra, so that the better and thus preferred library spectra can be searched against, in the future. Alternatively, if an earlier version of the profile mode library spectra has already been collected, a comparison can be made in terms of signal to noise and purity by using one or more of the library search score, accurate mass, spectral accuracy, retention index, and fragment analysis to decide whether the older version of the profile mode spectra should be replaced or retained in the library for future searches. The implementation via a Web or cloud server is expected to quickly evolve the inaccurate centroid-converted library into the more accurate profile mode mass spectral library. During the few minutes while the end user is awaiting for data uploading and/or analysis results, paid advertisements could be displayed to generate advertising revenues to fund the Web or cloud business operations. The advertisements could even be tailored to the type of compounds being detected to make the display ads even more relevant and effective.
   X. The actual test sample data may come from a variety of different instrumentations, such as Agilent GC/MSD, Thermo Fisher GC/ISQ, and Shimadzu GCMS-QP Series, instruments designed with different ion sources, ion optics, analog or digital electronics etc. These data typically are not directly comparable in raw profile mode, each with their own MS calibration and unique MS peak shapes which are also functions of the MS tune used to acquire the data. While the profile mode library data thus created would still be useful, they would not be as accurate, without a comprehensive MS calibration including MS peak shape, e.g., using the approach described in U.S. Pat. No. 6,983,213. It is of particular importance to specify the target MS peak shape function to be exactly the same across all samples measured across all MS instruments, which would ensure that the sample compound after the comprehensive MS calibration provide exactly the same accurate mass and spectrally accurate profile mode mass spectra, subject only to random noise, an overall scale difference due to ionization efficiency, or a specific scale difference due to a particular fragment ion produced from a molecular ion on a particular MS system. Such accurate mass and spectrally accurate profile mode mass spectra would not only allow for accurate compound search in the library for qualitative analysis, it would also enable both qualitative and quantitative analysis of a mixture of compounds that are either hard to separate or elute at the exact same time, and thus require 2D GC or LC separation.
   xi. For practical purposes, the above mentioned accurate mass and spectrally accurate profile mode mass spectral library, or a hybrid with the centroid-converted profile mode library during the enhancement or creation process, can be digitally recreated and released with different target peak shape functions, e.g., one with a Gaussian shape of FWHM (Full-Width-at-Half-Maximum) at 0.50 Da and one with a Gaussian shape of FWHM=0.85 Da, so as to be suitable to MS systems of different resolutions.

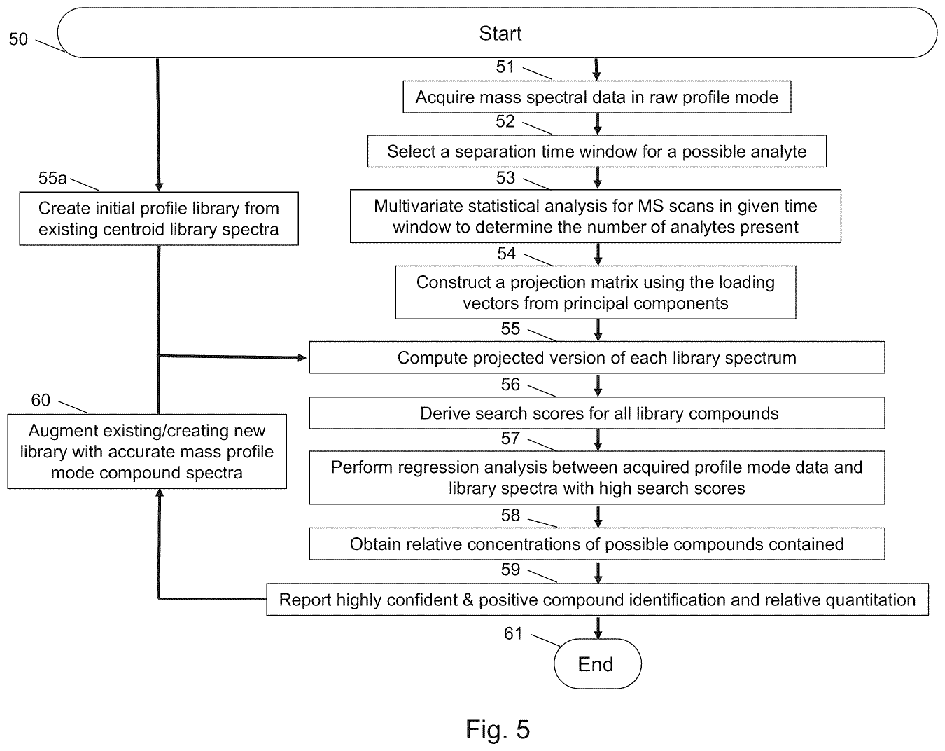

FIG. 5 shows the above steps in a flow chart of the embodiment described herein where at 51, mass spectral data is acquired in raw profile mode. At 52 a time window is selected corresponding to a detected peak from above step (a) so as to avoid analyzing a separation time window where no possible compounds are found. On the other hand, when computing power is not a concern, especially with modern computers, one may opt to segment a whole run into a series of time windows arranged one right after another to cover the whole separation time range, or to compute the entire separation time range as a single time window. At 53, multivariate statistical analysis for MS scans in a given time window is performed to determine the number of analytes present. At 54, a projection matrix is constructed based on the loading vectors of the principal component analysis performed above. At 55, the projected version of each library spectrum is computed. At 56, the corresponding search score is derived from the projected library spectrum. At 57, a regression between acquired profile mode data and the library spectral data with high search scores is performed to obtain the relative concentrations of corresponding possible compounds in 58. For the regression to work accurately, it is highly desirable to perform mass accuracy and spectral accuracy calibration of both the acquired profile mode sample data and all the profile mode library spectral data, using the approach initially described in U.S. Pat. No. 6,983,213 and specifying the same identical target peak shape function. At 59, highly confident compound identification and relative concentration results are reported with high quality accurate mass profile mode spectra added into the spectral library for future use and possible replacement of older less accurate data (Step 60). At 55a, independent of actual real sample analysis, an initial profile mode library can be created by convoluting a given peak shape function with the centroid mode spectral library already in existence, to help jump start the AMPS library and search.

AMPS can optionally work with accurate mass centroid data now available with GC TOF or GC Orbitrap MS, by converting accurate mass centroids into profile spectra through convolution with a specific peak shape, an operation which does not materially slow the search. AMPS can be used for any sort of MS data, integer centroids, accurate mass centroids, or full profile spectra, yet allows for higher-quality data if and when available.

In the above preferred embodiments, the chromatographic time profile calibration standards such as alkane with different carbon numbers could also serve as a retention time standard for the conversion of actual retention time into a retention index, which would allow for an additional dimension of compound identification by library search, since one could verify that the retention index calculated for an unknown compound also matches that of the library compound, in addition to a high library search score and high mass accuracy and spectral accuracy (SA). In fact, one could combine all these match scores to obtain an overall measurement of the match quality for compound identification. Similarly for compounds not already contained in the library (true unknowns) or compounds already contained in the library with missing, less accurate, or incorrect retention index data, this would allow the newly measured retention index to be created, added, or used to replace the less accurate or incorrect values.

An additional advantage of chromatographic retention index searches or matches is that the user can determine a set or range of possible compounds from a known compound library based on the retention index as computed for a chromatographic peak and its associated confidence interval (or error bar). This set or range of tentatively identified compounds may be completely overlapped with each other with little or no time separation, making reliable deconvolution statistically unstable or mathematically impossible. One may in this case perform a regression analysis described in U.S. Pat. No. 7,577,538 between the measured profile mode mass spectrum and those constructed from a library for both qualitative analysis (identification) and quantitative analysis, using the regression coefficients as an indication of likely quantities and fitting statistics (e.g., t-values) as an indication of the likely presence of compounds. Such a combined quantitative and qualitative analysis can be made significantly more accurate with an accurate mass and spectrally accurate profile mode library and could potentially be a replacement for more expensive and complex 2D GC or LC separation systems. The regression coefficients can be related to the actual concentrations through a calibration curve built with standard concentration series to achieve absolute quantitation or semi-quantitative results by ratioing against other internal or external reference standards or ions.

In many MS instruments such as quadrupole MS, the mass spectral scan time is not negligible compared to the compound (volatile compound, protein or peptide) elution time. Therefore, a significant skew would exist where the ions measured in one mass spectral scan come from different time points during the LC elution, similar to what has been reported for GC/MS (Stein, S. E. et al, J. Am. Soc. Mass Spectrom. 5, 859 (1994)). It is preferred to correct for any time skew existing in a typical slow-scanning quadrupole chromatography/mass spectrometry system so as to assure that all masses are “acquired” at the same chromatographic retention time, regardless of scan rate or the actual time it takes to scan the designated mass range. This can be accomplished through interpolation of the actual acquisition time for each m/z location onto a grid of the same actual retention time, by taking into consideration the MS scan rate, scan direction (from low to high m/z, vice versa, or a combination) and the dwell time between two successive scans. This skew correction will improve the performance of multivariate statistical analysis such as multiple linear regression (MLR), Principal Component Analysis (PCA), Partial Least Squares (PLS) etc. for the determination of the correct number of components using mass spectral scans within a separation time window or a deconvolution analysis.

As is known for those in the art, the term mass spectral library means the same as mass spectral database, regardless of the types of compounds involved, whether they are small molecules such as pesticides or large biomolecules such as proteins or peptides.

Although the description above contains many specifics, these should not be construed as limiting the scope of the invention but as merely providing illustrations of some feasible embodiments of this invention.

Thus, the scope of the disclosure should be determined by the appended claims and their legal equivalents, rather than by the examples given. Although the present disclosure has been described with reference to the embodiments described, it should be understood that it can be embodied in many alternate forms of embodiments. In addition, any suitable size, shape or type of elements or materials could be used. Accordingly, the present description is intended to embrace all such alternatives, modifications and variances which fall within the scope of the appended claims.

It will be understood that the disclosure may be embodied in a computer readable non-transitory storage medium storing instructions of a computer program which when executed by a computer system results in performance of steps of the method described herein. Such storage media may include any of those mentioned in the description above.

The techniques described herein are exemplary and should not be construed as implying any particular limitation on the present disclosure. It should be understood that various alternatives, combinations and modifications could be devised by those skilled in the art. For example, steps associated with the processes described herein can be performed in any order, unless otherwise specified or dictated by the steps themselves. The present disclosure is intended to embrace all such alternatives, modifications and variances that fall within the scope of the appended claims.

The terms “comprises” or “comprising” are to be interpreted as specifying the presence of the stated features, integers, steps or components, but not precluding the presence of one or more other features, integers, steps or components or groups thereof.

What is claimed is:

1. A method for mass spectral analysis of a sample for the identification of the compounds of interest, comprising the steps of
a. acquiring mass spectral data for a sample in a profile mode;
b. computing spectral loadings from the acquired mass spectral data;
c. operating with the spectral loadings and a mass spectral library spectrum in profile mode to calculate a search score for a library compound; and
d. reporting search scores indicating the likelihood of library compounds being present in said sample.

2. The method of claim 1, where the sample has gone through a separation process before mass spectral detection.

3. The method of claim 2, where the technique for separation is one of gas chromatography (GC/MS), liquid chromatography (LC/MS), supercritical fluid chromatography, ion chromatography (IC/MS), capillary electrophoresis (CE/MS), gel electrophoresis, ion mobility, and pyrolysis.

4. The method of claim 1, where the mass spectrometer is one of a sector mass spectrometer, ion trap mass spectrometer, quadrupole mass spectrometer, Time-of-Flight (TOF) mass spectrometer, Orbitrap mass spectrometer, and Fourier-transform ion cyclotron resonance (FT ICR) mass spectrometer.

5. The method of claim 1, where the profile mode library spectra are calibrated with a known set of standard ions for at least one of mass accuracy and spectral accuracy with a specific peak shape.

6. The method of claim 1, where the acquired profile mode mass spectral data are calibrated with a known set of standard ions for at least one of mass accuracy and spectral accuracy with a specific peak shape.

7. The method of claim 6, where said specific peak shape is substantially the same as that used for library spectra.

8. The method of claim 1, wherein mass spectral profile mode data are taken from a relevant separation window and are analyzed through principal component analysis to determine the statistically significant number of compounds present.

9. The method of claim 8, where the principal component analysis can be performed through one of singular value decomposition and Nonlinear Iterative Partial Least Squares (NIPALS) algorithm.

10. The method of claim 1, where the spectral loadings are obtained from the principal components.

11. The method of claim 1, where a library spectrum is projected through a projection matrix composed of spectral loadings to obtain a projected library spectrum from which a search score is derived.

12. The method of claim 1, where dot products between a library spectrum and all spectral loadings are used as the coefficients to linearly combine with respective spectral loadings to form a projected library spectrum from which a search score is derived.

13. The method of claim 1, where the search scores for all compounds in a collected library are sorted with the high search scores indicating the likely compounds for consideration.

14. The method of claim 1, where likely compounds present are selected based on one of their retention times or retention index.

15. The method of claim 1, where the relative concentrations of likely compounds in said sample are obtained as regression coefficients between the acquired profile mode mass spectrum and the library spectra of the likely compounds present.

16. The method of claim 15, where statistical measures including the t-values of the estimated relative concentrations are also obtained from the regression analysis to indicate at least one of significance of estimated concentrations, presence, and absence of certain compounds.

17. The method of claim 15, where the relative concentrations of likely compounds obtained are for a plurality of points during a separation process to form the separation profiles of these compounds including chromatograms.

18. The method of claim 15, where the regression is a multiple linear regression through the use of one of matrix computation, matrix inversion, singular value decomposition, principal component analysis, and partial least squares.

19. The method of claim 15, where a quantitative analysis is performed using the obtained relative concentrations.

20. The method of claim 1, where existing centroid mass spectral library data are converted into profile mode spectral data by convoluting the centroid library data with a specific mass spectral peak shape to form the profile mode spectral library initially.

21. The method of claim 1, where said acquired profile mode mass spectral data after analysis and identification are added into the mass spectral library in profile mode to one of augment or replace existing spectral data in the library.

22. The method of claim 12, where the dot product computation is implemented using modern computer parallelism including SIMD (Single Instruction Multiple Data) instructions, GPUs, and multicore CPUs for speed.

23. The method of claim 1, where it is implemented in the form of a server centrally located amongst a network including computers, devices, MS instruments, intranet or internet.

24. The method of claim 1, wherein users of the method are provided with a subscription to updates in the library.

25. A mass spectrometer operating in accordance with the method of claim 1.

26. For use with a computer associated with a mass spectrometer, a computer readable medium having computer readable program instructions readable by the computer for causing the mass spectrometer to operate in accordance with the method of claim 1.
